(19) United States
(12) Patent Application Publication
(10) Pub. No.: US 2001/0053781 A1
(43) Pub. Date: Dec. 20, 2001

(54) Pharmaceutical composition for enhancing cognition

(75) Inventors: Ming-Tsuen Hsieh, Taichung (TW); Ying-Tsung Lin, Nantou Hsien (TW)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: National Science Council

(21) Appl. No.: 09/791,958

(22) Filed: Feb. 22, 2001

(30) Foreign Application Priority Data
Jun. 16, 2000 (TW) 89111850

(51) Int. Cl. A61K 31/4965; A61K 31/19

(52) U.S. Cl. 514/255.01; 514/570

(57) ABSTRACT

The invention discloses a pharmaceutical composition for enhancing cognition, preventing and/or treating cognition disorders, including 50-100 mg/kg ferulic acid or a pharmaceutically acceptable salt or derivative thereof, and a pharmaceutically acceptable carrier or excipient. The pharmaceutical composition is useful in the treatment of disorders of learning acquisition, memory consolidation, and retrieval.

### BACKGROUND OF THE INVENTION

1. Field of the Invention

The present invention relates to a pharmaceutical composition for enhancing cognition, preventing and/or treating cognition disorders. More particularly, it relates to the pharmaceutical composition comprising ferulic acid and its derivatives as the active ingredient for enhancing cognition, preventing and/or treating cognition disorders.

2. Description of the Related Arts

Cognition disorders are wide problems in recent society and have potential danger to society. Therefore, scientists have made efforts in the development of cognition enhancers or cognition activators. The cognition enhancers or activators which have been developed are generally classified into 7 types, including nootropics, vasodilators, metabolic enhancers, psychostimulants, cholinergic agent, biogenic amines drugs, and neuropeptides. In these drugs, vasodilators and metabolic enhancers (e.g. dihydroergotoxine) are mainly effective in the cognition disorders induced by cerebral vessel ligation-ischemia; however, they are ineffective in clinical use and with other types of cognition disorders. In addition, cholinergic agent (e.g. tacrine), biogenic amines drugs, and neuropeptides can ameliorate learning and memory impairments of different magnitudes; however, they have not been formulated as an appropriate drug in clinical use⁽¹⁾. In regard to nootropics (e.g. “piracetam”, a drug proposed by Giurgea (1972), the meaning of which term refers to enhancing learning memory and promoting information convergence in brain⁽²⁾), various learning and memory impairments can be improved by nootropics, the mechanism of which involves the increasing of the turnover and firing rate of norepinephrine and dopamine in cerebral hippocampus and limbic system such as amygdala⁽³⁾. When the adrenal gland in rats is excised, the action of piracetam can be partially antagonized. It is therefore believed that piracetam functions via both a central and peripheral action⁽⁴⁾.

In the developed cognition enhancers, only metabolic drugs are employed for clinical use, others are still in the investigation stage, including piracetam, which exhibits uncertain effects on cognition disorders in clinical use. In addition, piracetam activates the action of peripheral endocrine, which is not appropriate for Alzheimer's disease due to the high concentration of steroids produced in patients. Another drug is tacrine, which became available in the market in 1993. Although the effect of tacrine is better than that of piracetam, more adverse side effects of tacrine appear, including vomiting, diarrhea, hepatotoxicity, etc. Therefore, the desired therapeutic effect of several cognition enhancers used in clinical applications are not attained.

Ferulic acid, 4-hydroxy-3-methoxycinnamic acid (CAS No. [1135-24-6]), is a known compound, which is present in various herbal medicines such as Angelica sinesis, Ligusticum chuanxiong, Ziziphus jujuba, etc. Therefore, ferulic acid is generally used as an indicator for testing the active ingredient present in many medical preparations containing the above herbal components. Wu and Yan (1996) disclose the method for quantification of ferulic acid in herbal medicine⁽⁵⁾; however, no prior art discloses the relationship between ferulic acid and cognition enhancement.

Bibliographic details of the publications numerically referred to in this specification are collected at the end of the description. All publications cited herein are incorporated by reference in their entirety.

### SUMMARY OF THE INVENTION

It is therefore a primary object of the present invention to provide a pharmaceutical composition for enhancing cognition, preventing and/or treating cognition disorders. The pharmaceutical composition comprises 50-100 mg/kg ferulic acid or a pharmaceutically acceptable salt or derivative thereof, and a pharmaceutically acceptable carrier or excipient.

### BRIEF DESCRIPTION OF THE DRAWINGS

The present invention will be more fully understood and further advantages will become apparent when reference is made to the following description of the invention and the accompanying drawings in which:

FIG. 1 is a diagram showing effects of ferulic acid (FA), piracetam (PIR), and tacrine (THA) on the scopolamine (SCOP)-induced acquisition impairment of passive avoidance response in rats.

FIG. 2 is a diagram showing effects of ferulic acid (FA), piracetam (PIR), and tacrine (THA) on the mecamylamine-induced acquisition impairment of passive avoidance response in rats.

FIG. 3 is a diagram showing effects of ferulic acid (FA) on the pirenzepine-induced acquisition impairment of passive avoidance response in rats.

FIG. 4 is a diagram showing effects of ferulic acid (FA), piracetam (PIR), and tacrine (THA) on the cycloheximide (CXM)-induced memory storage impairment of passive avoidance response in rats.

FIG. 5 is a diagram showing effects of ferulic acid (FA, 100 mg/kg) administered various times before or immediately after a trial of the cycloheximide (CXM)-induced memory storage impairment of passive avoidance response in rats.

FIG. 6 is a diagram showing effects of ferulic acid (FA, 100 mg/kg) on the scopolamine (SCOP, 0.3 mg/kg) plus mecamylamine (MECA, 0.3 mg/kg)-induced acquisition impairment of passive avoidance response in rats.

FIG. 7 is a diagram showing effects of ferulic acid (FA, 100 mg/kg) in combination with scopolamine methylbromide (M-SCOP, 0.5 mg/kg) on the scopolamine (SCOP)-induced acquisition impairment of passive avoidance response in rats.

FIG. 8 is a diagram showing effects of ferulic acid (FA) on the AF64A-induced impairment of passive avoidance response in rats.

FIG. 9 is a diagram showing effects of ferulic acid (FA, 50 and 100 mg/kg) on the deficit of place learning in the Morris water maze task induced by scopolamine (SCOP, 0.3 mg/kg) in rats. 10 rats were used in each group. *p<0.05, **p<0.01, compared with SCOP.

FIG. 10 is a diagram showing effects of ferulic acid (FA) on the cerebral blood flow rate measured by laser Doppler flowmetry in rats.

### DETAILED DESCRIPTION OF THE INVENTION

In general, learning and memory processes such as acquisition, memory consolidation/storage and retrieval⁽⁶⁾ are related to modification of neurotransmitters such as acetylcholine, dopamine, serotonin and synthesis of new protein. Recently, according to biochemical and electro-physiological studies, the things that will affect those memory processes are as follows:

(A) The sensitivity of postsynaptic membrane to acetylcholine increases after learning, thereby resulting in memory formation; however, when the sensitivity decreases or the membrane receptor is blocked, amnesia occurs⁽⁷⁾. In the learning acquisition process, the cholinergic neuronal system plays an important role with humans and animals. Scopolamine (SCOP) is a muscarinic antagonist, decreasing cholinergic activity and impairing learning and memory in rodents and human, especially in learning acquisition⁽⁸⁾. In addition, mecamylamine (MECA) is a nicotinic receptor blocker, which has a similar effect of acquisition impairment to SCOP. Therefore, drugs such as SCOP and MECA are used to induce the impairment of learning acquisition.

(B) For memory consolidation/storage processes, brain protein synthesis is an essential step followed by various forms of training. Cycloheximide (CXM) is a protein synthesis inhibitor that impairs memory consolidation and long-term memory in rodents⁽⁹⁾. In addition, some reports suggest that the close relationship between memory consolidation, which is impaired by CXM, and neurotransmitters⁽¹⁰⁻¹²⁾. Therefore, CXM-induced impairment appears to be a useful model for evaluating the mechanisms of the ameliorating drugs on memory consolidation.

The object of the present invention is to investigate the effects of ferulic acid and the derivatives thereof on the above-described drug-induced cognition disorders.

Therefore, the present invention provides a pharmaceutical composition for enhancing cognition, preventing and/or treating cognition disorders. The pharmaceutical composition comprises 50-100 mg/kg ferulic acid or a pharmaceutically acceptable salt or derivative thereof, and a pharmaceutically acceptable carrier or excipient.

In accordance with the present invention, the related derivatives of ferulic acid are also within the scope of the present invention. The derivatives include, but are not limited to, amino alcohol ester, dimeric ether, piperazine, or ester of ferulic acid; wherein the ester derivatives include, for example, methyl, ethyl, propyl, or isobutyl ferulate. The syntheses of such compounds and derivatives are well known to those skilled in this art belonging to organic chemical or chemical synthesis. It has been known that these derivatives have the same therapeutic effects to ferulic acid on the physiology or treating disorders (for example, enhancing immunity, scavenging and/or suppressing free radicals, treating ischemic apoplexy, coronary diseases, etc.)⁽¹³⁾.

As used herein, the pharmaceutically acceptable salts include salts with inorganic acids, such as hydrochloride, hydrobromide, sulfate and phosphate; salts with organic acids, such as acetate, maleate, tartrate, methanesulfonate; salts with amino acids, such as arginine, aspartic acid and glutamic acid; and salts with bases such as sodium hydroxide. Suitable pharmaceutical forms include sterile aqueous solutions or dispersions, sterile powders, tablets, troches, pills, capsules and the like. In addition, the active compounds may be incorporated into sustained-release preparations and formulations. The pharmaceutically acceptable carrier includes any and all solvents, disintegrating agents, binders, excipients, lubricants, absorption delaying agents and the like.

The pharmaceutical composition according to the present invention is useful in the enhancement of cognition, prophylaxis and/or treatment of cognition disorders, wherein cognition disorders include, but are not limited to, disorders of learning acquisition, memory consolidation, and retrieval, as described above.

Without intending to limit it in any manner, the present invention will be further illustrated by the following examples.

### EXAMPLE

### Example 1

### Experimental Design

1. Experimental Animals

Male Sprague-Dawley rats, weighing 200-250 grams, were housed in groups of six with free access to food and water and kept in a regulated environment (23±1° C.), wherein a 12-hour light-dark cycle (08:00-20:00 hours light) was maintained. Each experimental group included 12-18 rats.

2. Passive Avoidance Task

Rats were trained in a step-through passive avoidance task (Muromachi Kikai Co. Ltd. Japan). The apparatus consisted of two compartments having a steel-rod grid floor (36 parallel steel rods, 0.3 cm in diameter set 1.5 cm apart). One of the compartments (48×20×30 cm) was equipped with a 20 Watt lamp located centrally at a height of 30 cm, and the other was a dark compartment of the same size connected through a guillotine door (5×5 cm). The dark room was used during the experimental sessions that were conducted between 09:00 and 17:00.

3. Passive Avoidance Test

During the trial training, the guillotine door connecting the light and dark compartment was kept closed. After each rat was placed in the light compartment with its back to the guillotine door, the door was opened and simultaneously the time (step-through latency; STL) taken by the rat to enter the dark compartment was measured with a stopwatch⁽¹⁴⁾. Once the rat entered the dark compartment, the door was closed. An inescapable scrambled footshock (1.0 mA for 2 sec) was then delivered through the grid floor. The rat was removed from the dark compartment 5 seconds after administering the shock. The rat was then put back into its home cage until the retention trial, which was carried out 24 hours later. The rat was once again placed in the light compartment and as in the case of the training trial, the guillotine door was opened and the step-through latency was recorded and used as a measure of retention. An upper cut-off time of 300 seconds was set.

### Example 2

### Effects of Ferulic Acid on the Drug-Induced Cognition Disorders of Passive Avoidance Response in Rats

Ferulic acid (Sigma) was freshly dissolved in carboxymethyl cellulose, and administered to rats (50, 100 mg/kg, i.p.) 1 hour before the training trial in combination with the following drugs.

Drugs for inducing acquisition impairment: scopolamine HBr (muscarinic receptor blocker; 1 mg/kg, i.p.) was administered 30 minutes before the training trial⁽⁸⁾; pirenzepine (M₁ receptor blocker; 1 mg/kg, i.p.) was administered 30 minutes before the training trial⁽¹⁵⁾; and mecamylamine (nicotinic receptor blocker; 10 mg/kg, i.p.) was administered 30 minutes before the training trial⁽⁸⁾.

Drugs for inducing memory consolidation impairment: cycloheximide (protein synthesis inhibitor; 1.5 mg/kg, s.c.) was administered immediately after the training trial⁽⁹⁾.

The step-through latency of rats in light compartment was recorded according to the method described in Example 1. In the pathological control groups, drugs for inducing acquisition and memory consolidation impairments described above were administered. In the blank control group, vehicle (VEH; carboxy-methyl cellulose) was administered. In the positive control groups, piracetam (50 mg/kg, i.p., in physiological saline) and tacrine (3 mg/kg, i.p., in physiological saline) were administered 1 hour before the training trial, respectively.

### Example 3

### Effects of Central and Peripheral Neuron Systems on Ferulic Acid-Ameliorated Cognition Disorders

Ferulic acid (Sigma) was freshly dissolved in carboxymethyl cellulose, and administered to rats (50, 100 mg/kg, i.p.) 1 hour before the training trial in combination with the following drugs.

Drugs for inducing acquisition impairment: scopolamine (0.3 mg/kg, i.p.) and mecamylamine (3 mg/kg, i.p.) were co-administered 30 minutes before the training trial⁽⁸⁾.

Effects of peripheral neuron system: scopolamine (0.3 mg/kg, i.p.) and scopolamine methylbromide (M-SCOP, 0.5 mg/kg, i.p.) were co-administered 30 minutes before the training trial⁽¹⁶⁾.

Effects of central neuron system: AF64-A (central cholinergic neurotoxin, 3 nmol/μl/brain) was administered to the lateral ventricle 10 days before the training trial to destroy the cholinergic neuronal system in brain⁽¹⁷⁾.

The step-through latency of rats in light compartment was recorded according to the method described in Example 1. In the blank control group, vehicle (VEH; carboxy-methyl cellulose) was administered.

### Example 4

### Water Maze Test

The water maze apparatus (Columbus Instruments Intl. Co., USA) was used in this Example. The swimming time and track of rats in the water maze were monitored by the Videomex V System and recorded by computer software. The water maze apparatus consisted of a water tank of 180 cm in diameter and 35 cm in height. The water stained as milk-white color was poured into the tank (25 cm in depth, 23±1° C.) before the training trial. The tank was conceptually divided into the 4 quadrants of east, west, south and north on the computer. An acrylic plate of 10 cm in diameter was placed at the north quadrant 1 cm below the water level. Rats were trained 4 times per day as one training course for 5 days. In one training course, each rat swam to the acrylic plate from 4 different quadrants, respectively. 10 seconds after swimming to the acrylic plate, the rat was returned to the home cage. After 30 seconds of rest, the next training session was carried out. The swimming time from the beginning to the acrylic plate was recorded, and an upper cut-off time of 60 seconds was set⁽¹⁸⁾.

Ferulic acid (Sigma) was freshly dissolved in carboxy-methyl cellulose, and administered to rats (50, 100 mg/kg, i.p.) 1 hour before the training trial in combination with the following drug: scopolamine HBr (muscarinic receptor blocker; 0.3 mg/kg, i.p.) 30 minutes before the training trial for inducing acquisition impairment.

### Example 5

### Measurement of Cerebral Blood Flow Rate

The cerebral blood flow rate was measured by laser Doppler flowmetry in rats according to the procedure described in Morikawa, E. et al.⁽¹⁹⁾.

### Example 6

### Passive Avoidance Behavior with No Shock

Experimental steps were the same as those described in Example 1, but without the 1.0 mA footshock. 24 hours later, the retention trial was carried out. The rat was again placed in the light compartment, as in the training trial, the guillotine door was opened and the step-through latency was recorded and used as a measure of retention.

Results

Memory processes are divided into three stages: learning acquisition, memory consolidation, and retrieval. The “nerve connection” theory established by Ramon and Sherrington indicates that learning can increase the connection number of dendron and axon in brain. That is, the nervous system is malleable during the learning process. In addition, the presynaptic neurotransmitters are released resulting from learning, which cause the DNA replication, RNA transcription, and proteins synthesis in postsynaptic neurons and surrounding cells.

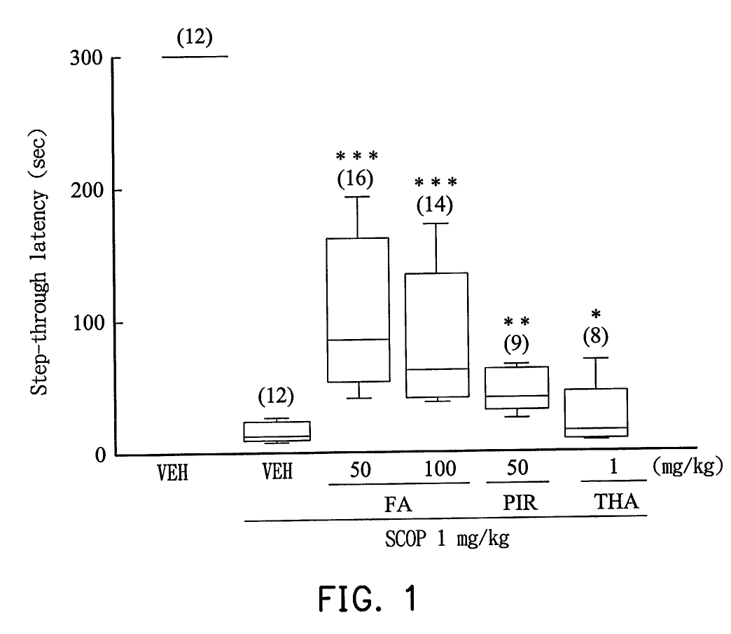

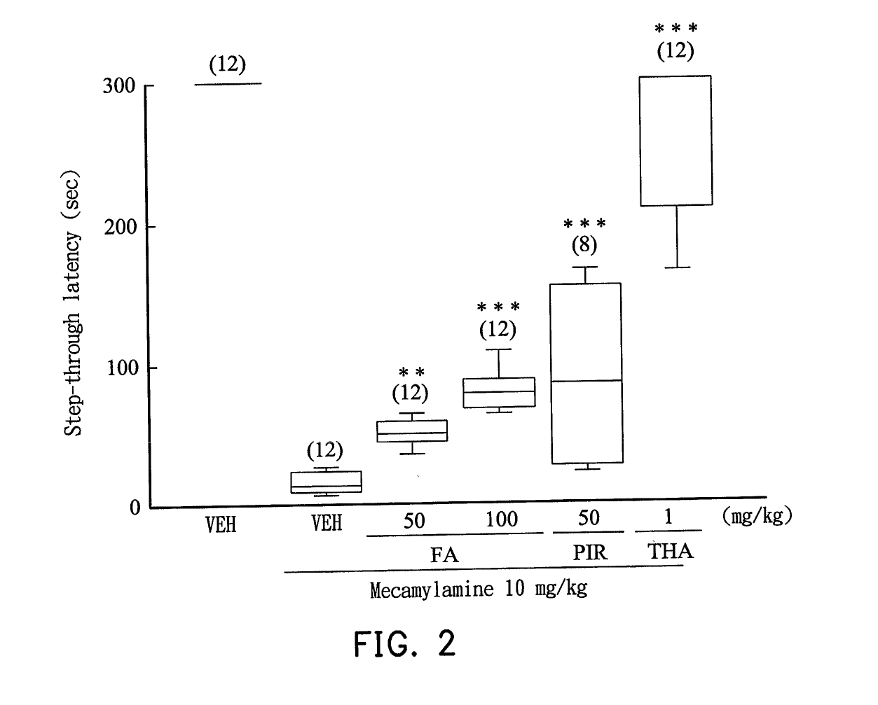

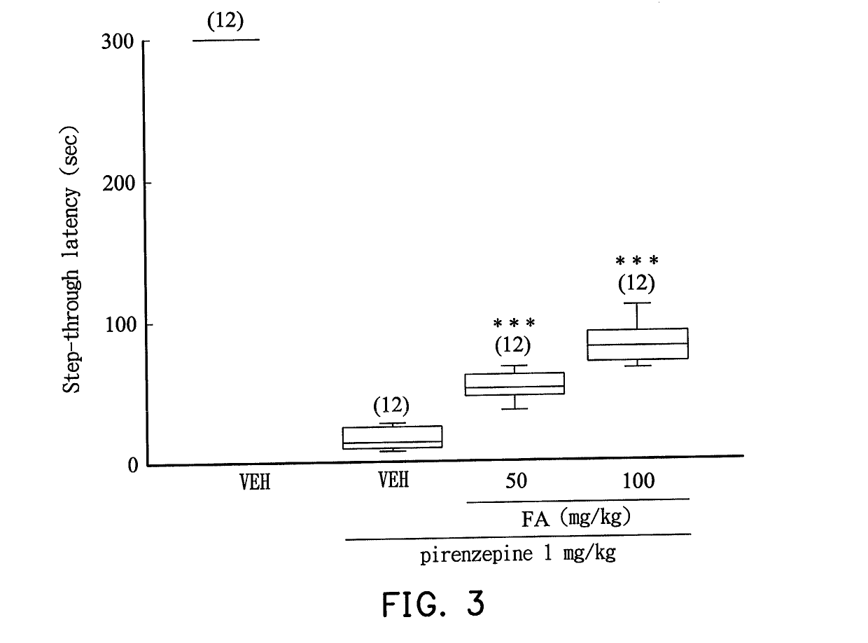

The sensitivity of postsynaptic membrane to acetylcholine increases during the learning process, thereby resulting in memory formation; however, when the sensitivity decreases or the membrane receptor is blocked, amnesia occurs. Therefore, the increase of cholinergic activity facilitates learning acquisition formation. On the contrary, the decrease of cholinergic activity facilitates learning acquisition impairment. Elrod (1988) suggested that the administration of scopolamine or mecamylamine can decrease the concentration of acetylcoline in the brain cortex, hippocampus and striatum, resulting in the decrease of cholinergic activity and causing anterograde memory damage and learning acquisition impairment. Thus, scopolamine and mecamylamine are used in the present invention as drugs for inducing learning acquisition. Referring to FIGS. 1-2, it is shown that ferulic acid at 50-100 mg/ml attenuates scopolamine- and mecamylamine-induced acquisition impairment. In addition, Worms, et al. (1989) reported the function of cholinergic neuronal system in brain is lost by acetylcholine M₁ receptor antagonist pirenzepine, resulting in anterograde memory damage and mainly affecting learning acquisition. Referring to FIG. 3, it is shown that ferulic acid at 50-100 mg/ml also counteracts pirenzepine-induced acquisition impairment.

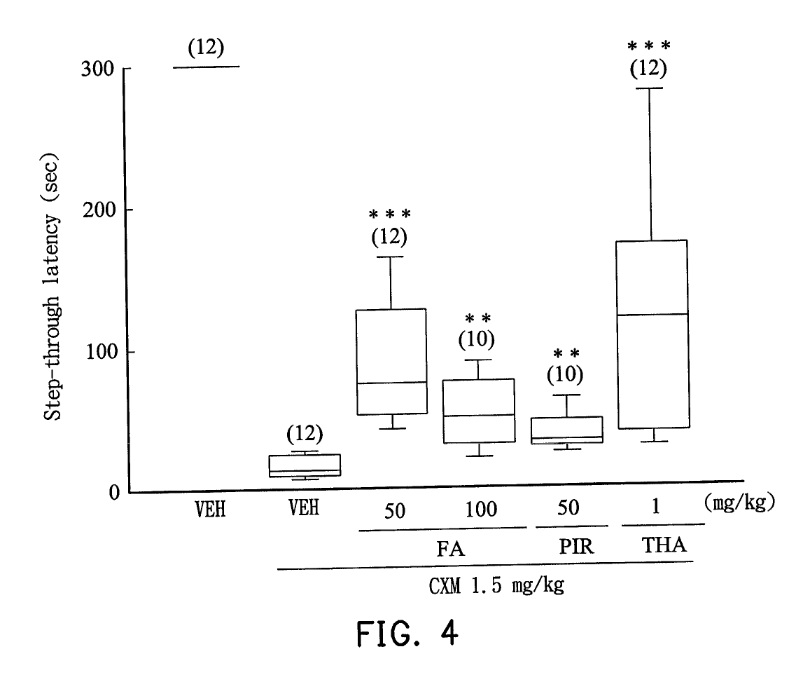

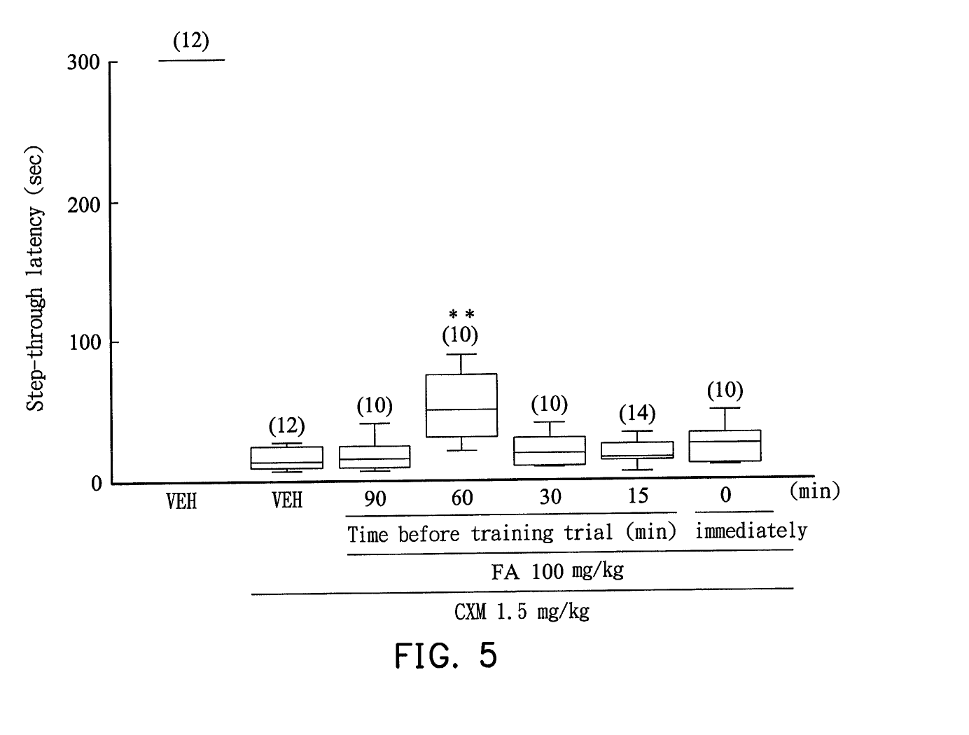

Protein syntheses play an important role in memory consolidation. The central nervous system is activated via external stimulation, causing the release of presynaptic neurotransmitters that act on the postsynaptic receptor. The enzymes present in the mitochondria and nucleus are thus activated by the secondary messengers such as cAMP, protein kinase C and nitric oxide, thereby increasing the synthesis of DNA, RNA, and proteins. This increases or changes the number of receptors on the membrane to regulate the corresponding response of stimulation, that is, the formation of memory consolidation. Cycloheximide (CXM) is a protein synthesis inhibitor and blocks about 80% of the protein synthesis in the central nervous system, which impairs memory consolidation. Referring to FIGS. 4-5, it is shown that ferulic acid at 50-100 mg/ml significantly ameliorates CXM-induced consolidation impairment, provided that ferulic acid is administered 60 minutes before the training trial.

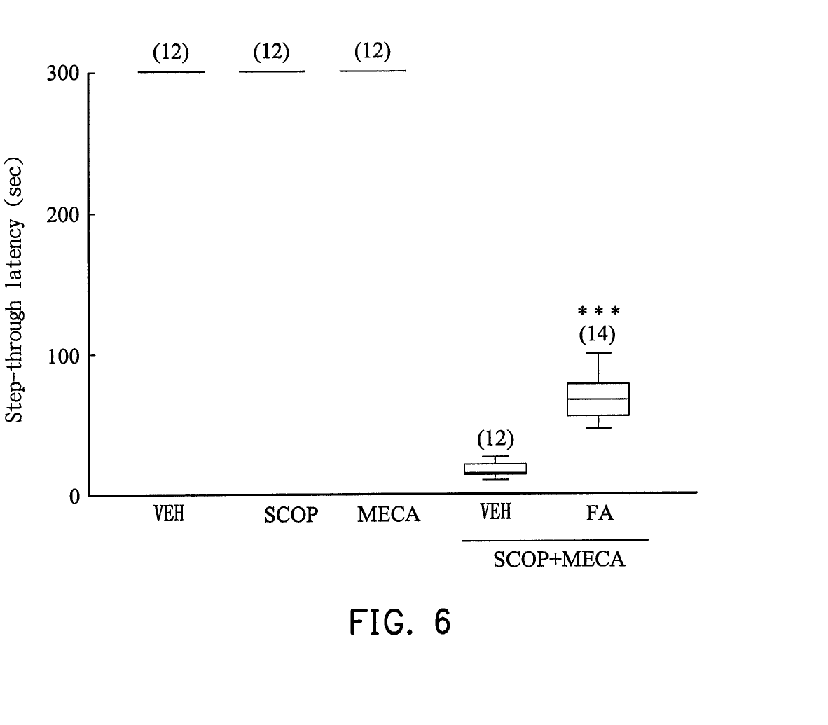

The cholinergic neuronal system plays an important role in the learning acquisition process, and both muscarinic and nicotinic receptors possess positive functions. Moreover, studies have shown the numbers of muscarinic and nicotinic receptors are remarkably decreased in aged people with cognition disorders, especially in the regions of brain cortex, hippocampus and nucleus basalis magnocellularis. Referring to FIG. 6, administration of both scopolamine and mecamylamine under the threshold dosage induces acquisition impairment. However, it is demonstrated that ferulic acid at 50-100 mg/ml attenuates the learning impairment induced by scopolamine plus mecamylamine, which does not cause the impairment of passive avoidance performance at used dosage alone.

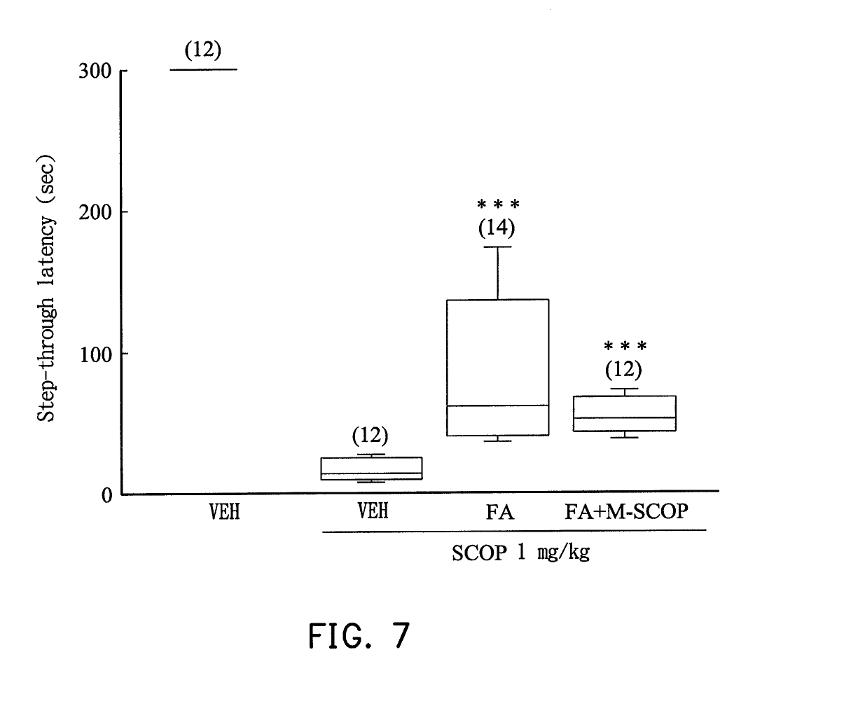

Theoretically, peripheral nervous system can regulate the response of central nervous system to the stimulation, and play a certain role in the learning acquisition process. It is found the function of cognition enhancer can be blocked by peripheral antagonists. In addition, some peripheral agents such as 4-hydroxy-amphetamine, neostigmine, and des-Tyr-D-Pro-casomorphin that cannot permeate the cerebral vessels are effective in ameliorating cognition, and their functions can be blocked by scopolamine methylbromide (peripheral antagonist). Referring to FIG. 7, ferulic acid at 100 mg/kg ameliorates scopolamine-induced acquisition impairment, but the peripheral cholinergic antagonist scopolamine methylbromide does not block the counteracting effects of ferulic acid on the scopolamine-induced learning impairment.

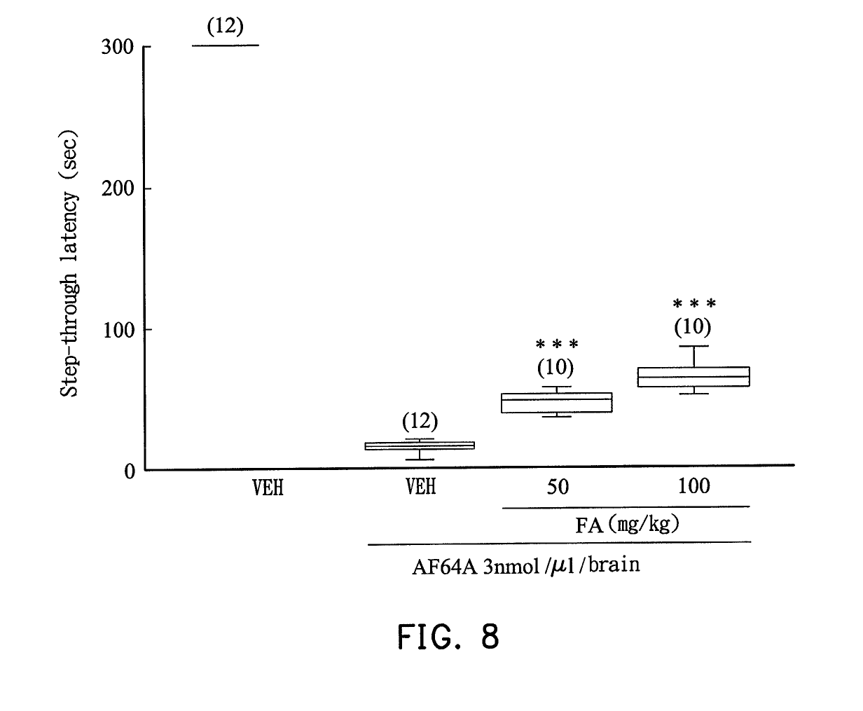

Deutsh (1971) showed the number of acetylcholine neuronal synaptic increases during message storage and retrieval. The central cholinergic neurotoxin AF64-A can not only damage and/or atrophy the acetylcholine neuron, but also impair learning acquisition. Referring to FIG. 8, ferulic acid at 50-100 mg/kg counteracts the memory impairment induced by AF64A.

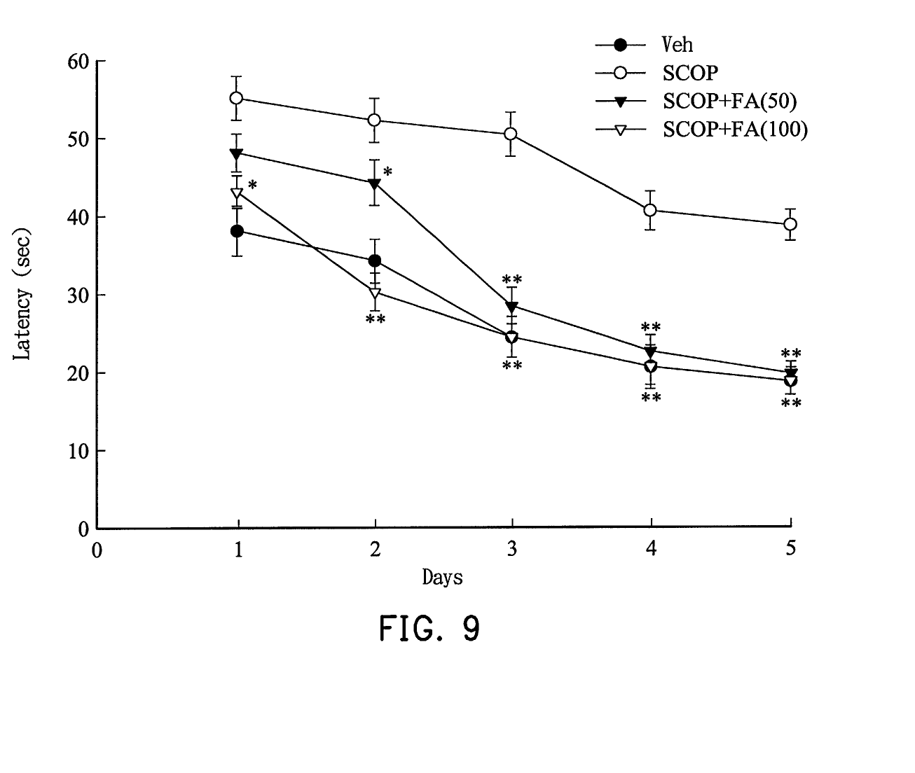

In addition to the simple model of passive avoidance response, the present invention employs a complex model, such as a water maze that involves the higher nervous system and evaluates the relationship between learning acquisition and drugs. Scopolamine is administered to rats before a training trial to induce acquisition impairment. Referring to FIG. 9, 50-100 mg/kg ferulic acid ameliorates scopolamine-induced acquisition impairment in a water maze.

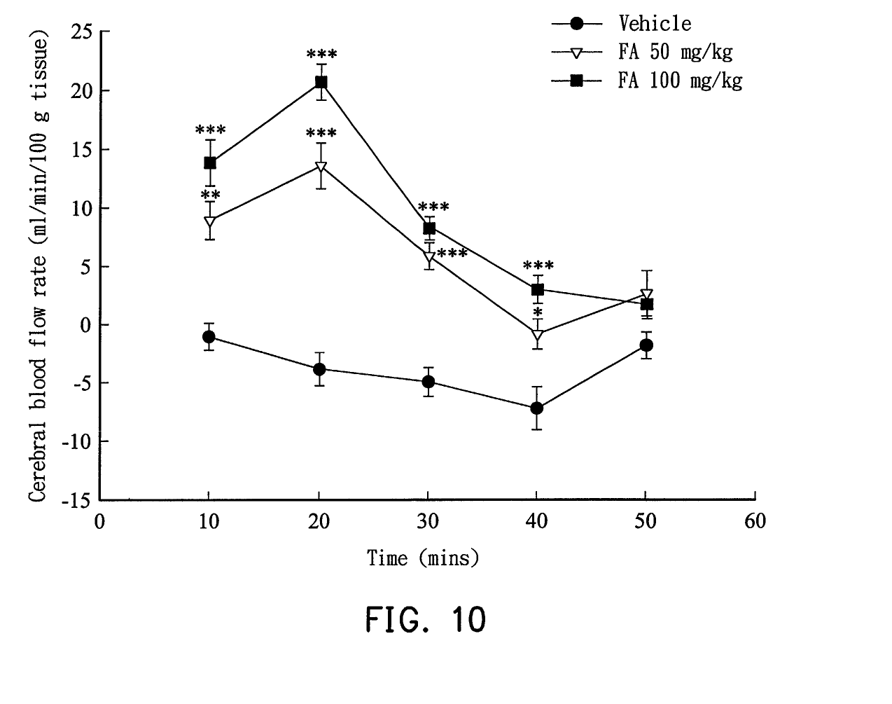

Aged people suffer from cognition impairment mainly because: (1) insufficient cerebral blood flow; and (2) insufficient supply of oxygen and glucose, and accumulation of metabolites. The developed cognition enhancers used in clinical application include co-dergocrine, nicergoline, pentoxifylline, etc. All of them belong to the drugs for improving the cerebral blood flow and metabolism. Referring to FIG. 10, it is demonstrated that 50-100 mg/kg ferulic acid enhances the cerebral blood flow rate from 10 to 40 minutes after administration.

Further, Xu et al. reported the half lethal dose (LD₅₀) of ferulic acid administered intravenously is 866±29 mg/kg⁽²⁰⁾, much higher than the effective dosage used in the present invention.

From the examples and data shown above, the present invention demonstrates the attenuating mechanism of ferulic acid on learning and memory impairment (amnesia).

While the invention has been particularly shown and described with the reference to the preferred embodiment thereof, it will be understood by those skilled in the art that various changes in form and details may be made without departing from the spirit and scope of the invention.

### REFERENCES

1. Moos, W. H., Davis, R. S., Schwarz, R. D. and Gamzu, E. R. (1988) Cognition activators. Med. Res. Rev. 8: 353-391.

2. Giurgea, C. (1972) Vers une pharmacologie de l'activite' int'egrative ducerveau. Tentative ddu concept nootrope en psychopharmacologie. Actual Pharmacol 25: 115-156.

3. Kov'as, G. L., Dirk, H. G., Versteeg, E., Ronald, de K. and Be'la B. (1981) Passive avoidance performance correlates with catecholamine turnover in discrete limbic brain regions. Life Sci. 28: 1109-1116.

4. Gouliaev, A. H. and Senning, A. (1994) Piracetam and other structurally related nootropics. Brain Res. Rev. 19: 180-222.

5. Wu, N. F. and Yan, C. G. (1996), J. China Pharmacy, 7: 278.

6. Izquierdo, I. (1984) Endogenous state dependency: memory depends on the relation between the neurohumoral and hormonal states present after training and at the time of testing. In: G. Lynch, J. L. McGaugh and N. M. Weinberger (Eds.), Neurobiology of learning and memory, The Guilford Press, New York, pp.333-350.

7. Deutsch, J. A. (1971) The cholinergic synapse and the site of mechanism. Science 174: 788-794.

8. Elrod, K. and Buccafusco J. J. (1988) An evaluation of the mechanism of scopolamine-induced impairment in two inhibitory avoidance protocols. Pharmacol. Biochem. Behav. 29: 15-21.

9. Davis, H. P. and Squire L. R. (1984) Protein synthesis and memory: a review. Psychol. Bull. 96: 518-559.

10. Nabeshima, T., Itoh, K., Kawashima, K. and Kameyama, T. (1989) Effects of 5-HT2 receptor antagonist on cycloheximide-induced amnesia in mice. Pharmacol. Biochem. Behav. 32: 787-790.

11. Nabeshima, T., Tohyama, K., Murase, K., Ishihara, S., Kameyama, T., Yamasaki, S., Hatanaka, S., Kojima, H., Sakurai, T., Takasu, Y. and Shiotani, T. (1991) Effects of DM-9384, a cyclic derivative of GABA, on amnesia and decreases in GABA_{A} and muscarinic receptors induced by cycloheximide. J. Pharmacol. Exp. Ther. 257: 271-275.

12. Nabeshima, T., Marnyama, B., Katoh, A. and Kameyama T. (1991) The effect of tacrine (THA) on cycloheximide- and basal forebrain lesion-induced memory deficit in rats. Jpn. J. Pharmacol. 57: 311-319.

13. Lin I. H. and Chen W. W. (1994), Acta Pharmaceutica Sinica 29(9):717-720.

14. Hsieh, M. T., Wu, C. R. and Hsieh C. C. (1998) Ameliorating effect of p-hydroxybenzyl alcohol on cycloheximide-induced impairment of passive avoidance response in rats: Interactions with compounds acting at 5-HTIA and 5-HT₂ receptors. Pharmacol. Biochem. Behav. 60: 345-354.

15. Worms, P., Gueudet, C., P'erio, A. and Soubrie', P. (1989) Systemic injection of pirenzepine induced a deficit in passive avoidance learning in rats. Psychopharmacology 98: 286-288.

16. Peng, W. H., Hsieh, M. T. and Wu, C. R. (1997) Effect of long-term administration of berberine on scopolamine-induced amnesia in rats. Jpn. J. Pharmacol. 74: 261-266.

17. Abe, B., Murai, S., Masuda, Y., Saito, H. and Itoh, T. (1993) α-Sialyl cholesterol reveres AF64A-induced deficit in passive avoidance response and depletion of hippocampal acetyicholine in mice. Br. J. Pharmacol. 108: 387-392.

18. Markowska, A. L., Long, J. M., Johnson, C. T. and Olton, D. S. (1993) Variable-interval probe tests as a tool for repeated measurements of spatial memory in the water maze. Behav. Neurosci. 107: 627-632.

19. Morikawa, E., Zhang, S. M., Yoshinori, S., Tomikatsu, T., Takaaki, K. (1996) Treatment of focal cerebral ischemia with synthetic oligopeptide corresponding to lectin domain of selectin. Stroke 27: 951-955.

20. Xu, J., Li, Y., Liang, E. (1992) Effect of tetramethylpyrazine and ferulic acid alone or combined on vascular smooth muscle, blood viscosity and toxicity. China J. Chinese Materia Medica, 17(11): 680-682, 703-704.

What is claimed is:

1. A pharmaceutical composition for enhancing cognition, comprising:
(a) 50-100 mg/kg ferulic acid or a pharmaceutically acceptable salt or derivative thereof; and
(b) a pharmaceutically acceptable carrier or excipient.

2. A pharmaceutical composition for the prophylaxis or treatment of cognition disorders, comprising:
(a) 50-100 mg/kg ferulic acid or a pharmaceutically acceptable salt or derivative thereof; and
(b) a pharmaceutically acceptable carrier or excipient.

3. The pharmaceutical composition as claimed in claim 2, wherein cognition disorders comprise disorders of learning acquisition, memory consolidation, and retrieval.

4. The pharmaceutical composition as claimed in claim 1 or 2, wherein the derivative comprises amino alcohol ester, dimeric ether, piperazine, or ester of ferulic acid.

5. The pharmaceutical composition as claimed in claim 4, wherein the ester derivative comprises methyl, ethyl, propyl, or isobutyl ferulate.

6. A method for enhancing cognition in a mammal, comprising administering to said mammal 50-100 mg/kg ferulic acid or a pharmaceutically acceptable salt or derivative thereof.

7. A method for the prophylaxis or treatment of cognition disorders in a mammal, comprising administering to said mammal 50-100 mg/kg ferulic acid or a pharmaceutically acceptable salt or derivative thereof.

8. The method as claimed in claim 7, wherein cognition disorders comprise disorders of learning acquisition, memory consolidation, and retrieval.

9. The method as claimed in claim 6 or 7, wherein the derivative comprises amino alcohol ester, dimeric ether, piperazine, or ester of ferulic acid.

10. The method as claimed in claim 9, wherein the ester derivative comprises methyl, ethyl, propyl, or isobutyl ferulate.
